(19) United States
(12) Patent Application Publication
(10) Pub. No.: US 2009/0048449 A1
(43) Pub. Date: Feb. 19, 2009

(54) RIMONABANT MONOHYDRATE, PROCESS FOR THE PREPARATION THEREOF AND PHARMACEUTICAL COMPOSITIONS CONTAINING SAME

(75) Inventors: Gerard COQUEREL, Paris (FR); Helene DUPLAA, Paris (FR); Baptiste FOURS, Paris (FR); Olivier MONNIER, Paris (FR); Philippe OCHSENBEIN, Paris (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/186,753

(22) Filed: Aug. 6, 2008

Related U.S. Application Data
(63) Continuation of application No. PCT/FR2007/000201, filed on Feb. 5, 2007.

(30) Foreign Application Priority Data
Feb. 8, 2006 (FR) 0601253

(51) Int. Cl. C07D 401/02

(52) U.S. Cl. 546/211

(57) ABSTRACT

The present invention relates to rimonabant monohydrate, to the process for preparing it and to the pharmaceutical compositions containing it.

This application is a continuation of International application No. PCT/FR2007/000,201, filed Feb. 5, 2007, which is incorporated herein by reference in its entirety; which claims the benefit of priority of French Patent Application No. 06/01,253, filed Feb. 8, 2006.

The subject of the present invention is rimonabant monohydrate, its preparation method and the pharmaceutical compositions containing it.

Rimonabant is the international common name of N-piperidino-5-(4-chlorophenyl)-1-(2,4-dichlorophenyl)-4-methylpyrazole-3-carboxamide.

This compound, its salts and its solvates are described in the European Patent 656 354.

A polymorphic crystal form of rimonabant called “form II” is described in International Patent WO 2003/040105.

One particular solvate has now been found, namely rimonabant monohydrate that has advantageous properties.

### BRIEF DESCRIPTION OF THE DRAWINGS

FIG. 1 shows TGA and DSC thermograms of rimonabant monohydrate.

FIG. 2 shows DSC thermogram of rimonabant crystal form II.

FIG. 3 shows DSC thermogram of rimonabant monohydrate crystal form.

FIG. 4 shows sorption/desorption isotherm at 25° C. for the rimonabant monohydrate crystal form.

FIG. 5 shows IR spectrum of the rimonabant monohydrate crystal form.

FIG. 6 shows IR spectrum of the rimonabant crystal form II.

FIG. 7 shows powder X-ray diffractogram of the rimonabant monohydrate crystal form.

FIG. 8 shows powder X-ray diffractogram of the rimonabant crystal form II.

FIG. 9 shows the superposition of the theoretical (bottom line) and experimental (top line) X-ray diffractogram of the rimonabant monohydrate crystal form.

FIG. 10 shows the hydrogen bonds in the crystal lattice of the rimonabant monohydrate crystal form.

FIG. 11 shows powder X-ray diffractogram of the rimonabant monohydrate crystal form obtained in Example 1.

The term “rimonabant monohydrate” is understood to mean the chemical compound made from one molecule of rimonabant and one molecule of water.

Rimonabant monohydrate preferentially exists in a crystallized form. The present invention relates to rimonabant monohydrate, and more particularly to a crystal form of rimonabant monohydrate.

The fact of obtaining a rimonabant solvate with one molecule of water is particularly advantageous as rimonabant monohydrate constitutes an active principle that can be administered to man.

The crystal form of rimonabant monohydrate consists of a powder whose characteristics are improved compared with the powders consisting either of rimonabant in crystal form I or of rimonabant in crystal form II.

Thus, during separation of the rimonabant monohydrate crystals by filtering from the solution in which they were formed, surprisingly better filterability is observed than when the rimonabant form I crystals or form II crystals are filtered. The improvement in filterability allows the filtration step to be shortened and leads to a significant improvement in the texture of the filter cake which is characterized by a low moisture content of the powder before drying and a low degree of residual solvent before drying. The resulting powder after drying has improved physical properties, especially in terms of flowability and therefore of handleability.

The improvement in the filterability is measured by studying the characteristics of the filter cake: for the rimonabant monohydrate in crystal form, it is observed that it has a specific resistance less than that measured for rimonabant crystal form I and crystal form II.

The flowability of the rimonabant monohydrate crystal form was measured and compared to that of the rimonabant crystal form II. The flowability of the crystal forms is measured by the flowability index or compressibility index or Carr index as described in R. L. Carr: Evaluation of flow properties of solids, Chem. Eng., 1965, 163-168 and also in the European Pharmacopeia.

The flowability index is calculated according to the following equation: IC=100×(ρt−ρb)/ρt where ρt is the tap density and ρb is the bulk density. This index is considered to be good if it is less than 20.

The densities are determined experimentally by packing the product into a graduated cylinder according to the procedure described in the European Pharmacopeia. The densities are determined after 10, 500, 1250 and 2500 taps. The Carr index is determined from the data measured at 10 and 1250 taps.

A Carr index less than or equal to 20% is considered as corresponding to a good powder flow, whereas a Carr index greater than 21% is considered as corresponding to a passable, even difficult or very difficult, powder flow.

For the rimonabant monohydrate crystal form, a Carr index equal to 20%, that is to say equal to good powder flow, is measured, while for the rimonabant crystal form II, a Carr index of around 38%, that is to say equal to very difficult powder flow, is measured.

The Carr index measured for the rimonabant crystal form I also corresponds to a very difficult flowability.

The good flowability index of the rimonabant monohydrate crystal form indicates that this form could easily be mixed with excipients during the preparation of pharmaceutical compositions for administering rimonabant monohydrate. In particular, during the preparation of tablets, the flow of the powder is improved and the content of the active principle is better controlled. Due to the better flowability, the method for manufacturing tablets may be simplified by eliminating certain steps such as wet granulation, drying and sizing, which allows the production rates to be increased and the cost of production to be decreased.

The present invention also relates to the method for obtaining the rimonabant monohydrate. This method is characterized in that the rimonabant is dissolved in an organic solvent and water is added. More particularly, this method is characterized in that:

a) a mixture of rimonabant is prepared in a solvent chosen from:
methylcyclohexane;
acetonitrile;
4-methyl-2-pentanone;
acetone;
toluene;
dimethylsulfoxide; or
a mixture of these solvents;
   b) water is added drop by drop.

More particularly, in step a) a solvent is used chosen from:
methylcyclohexane;
acetonitrile;
4-methyl-2-pentanone;
acetone; or
a mixture of these solvents.

Preferentially, according to the method of the invention, step a) is carried out at room temperature.

Particularly, the method for preparing rimonabant monohydrate according to the invention is characterized in that:

a) a saturated solution of rimonabant is prepared in a solvent chosen from:
methylcyclohexane;
acetonitrile;
4-methyl-2-pentanone;
acetone;
toluene;
dimethylsulfoxide; or
a mixture of these solvents;
   b) Water is added drop by drop;
   c) the rimonabant monohydrate formed is separated.

More particularly, in step a) a solvent is used chosen from:
methylcyclohexane;
acetonitrile;
4-methyl-2-pentanone;
acetone; or
a mixture of these solvents;

Preferentially, after step a), the solution is filtered to obtain a saturated clear solution.

The rimonabant monohydrate formed by the method according to the invention is separated by filtration.

Particularly, in step a), a solution of rimonabant in acetone is prepared. More particularly, a solution containing between 150 and 200 g/l of rimonabant in acetone, and preferentially a solution containing 200 g/l of rimonabant in acetone, is prepared.

Particularly, in step b) water is added drop by drop so as to obtain an acetone/water mixture containing between 10 and 30% of water by volume; preferentially, the mixture contains 20% of water.

A method for obtaining the rimonabant monohydrate in crystal form is characterized in that:

a) a mixture of rimonabant is prepared in a solvent chosen from:
methylcyclohexane;
acetonitrile;
4-methyl-2-pentanone;
acetone;
toluene;
dimethylsulphoxide; or
a mixture of these solvents;
   b) water is added drop by drop;
   c) it is cooled to between 0° and 15° C.; and
   d) the crystals formed are filtered.

More particularly, in step a) a solvent is used chosen from:
methylcyclohexane;
acetonitrile;
4-methyl-2-pentanone;
acetone; or
a mixture of these solvents;

Particularly, the method for preparing rimonabant monohydrate in crystal form is characterized in that:

a) a saturated solution of rimonabant is prepared at room temperature in a solvent chosen from:
methylcyclohexane;
acetonitrile;
4-methyl-2-pentanone;
acetone;
toluene;
dimethylsulphoxide; or
a mixture of these solvents;
   b) Water is added drop by drop;
   c) it is cooled to between 0° C. and 15° C.; and
   d) the crystals formed are filtered.

More particularly, in step a) a solvent is used chosen from:
methylcyclohexane;
acetonitrile;
4-methyl-2-pentanone;
acetone; or
a mixture of these solvents;

Preferentially, after step a) the solution is filtered to obtain a saturated clear solution.

More particularly, the rimonabant monohydrate may be prepared in crystal form according to a method characterized in that:
a) a mixture containing between 150 and 200 g/l, preferentially 200 g/l, of rimonabant in acetone is prepared at room temperature;
b) between 10% and 30% of water by volume, preferentially 20% of water by volume, is added drop by drop;
c) it is cooled to a temperature between 0° C. and 15° C., preferentially 5° C.; and
d) the crystals formed are filtered.

After step a), the mixture formed may be filtered in order to obtain a saturated clear solution.

After the filtration in the last step, the product obtained is dried at a temperature between room temperature and 40° C., preferentially at room temperature.

Preferentially, the solvent used in step a) of the method according to the invention is acetone, which results in the rimonabant monohydrate being separated from an acetone/water mixture, this mixture has conductive properties and its use makes it possible to avoid the accumulation of electrostatic charges that are dangerous from an industrial viewpoint.

The rimonabant monohydrate is characterized by various components of its physico-chemical analysis.

Water Content:

The rimonabant monohydrate is characterized by elemental analysis and by analysis of the water content measured on a Karl Fisher apparatus.

Elemental analysis: C₂₂H₂₃O₂N₄Cl₃.

| | C | H | N |
|---|---|---|---|
| Theoretical | 54.84 | 4.81 | 11.63 |
| Measured | 55.07 | 4.83 | 11.50 |

The theoretical and measured values take into account the presence of one molecule of water.

Water content: measured: 3.7%±0.5%; calculated: 3.74%.

The water content indicates the presence of the equivalent of one molecule of water per molecule of product.

Thermogravimetry:

The thermogravimetric analysis was carried out for the rimonabant monohydrate by a TGA 2950 thermogravimetric analyzer, sold by TA Instruments SARL (Paris, France); it was operated under a nitrogen atmosphere, the initial temperature was 30° C., it increased at a rate of 10° C./minute until the decomposition of the product.

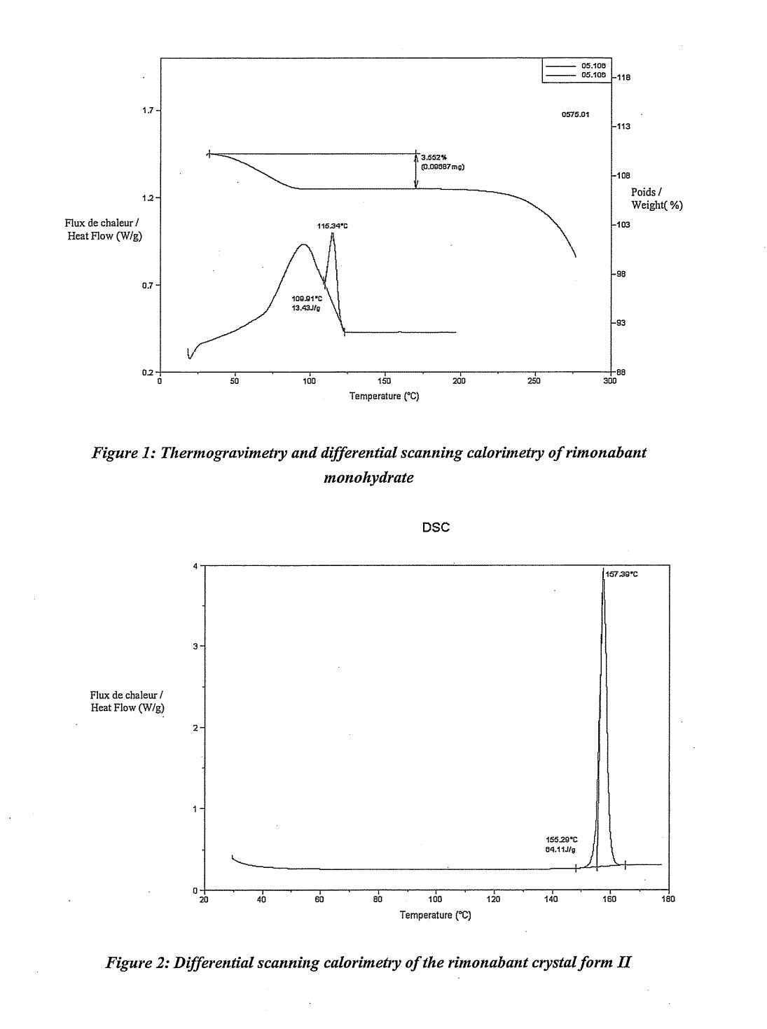

The theoretical weight loss corresponding to one mole of water is 3.74%. Experimentally, by thermogravimetric analysis, it is equal to 3.55%. This result is in agreement with theory and confirms that the product tested contains one molecule of water that disappears in the same temperature zone as in differential scanning calorimetry, namely between 40° C. and 100° C. (FIG. 1).

The thermogravimetry weight loss curve indicates that the water molecule present is a hydration molecule.

The crystal form of the rimonabant monohydrate was also analyzed and characterized.

Differential Scanning Calorimetry:

The differential scanning calorimetry of the rimonabant monohydrate crystal form was carried out under the same conditions on a MDSC 2920 differential scanning calorimeter, sold by TA Instruments SARL (Paris, France); it was operated under a nitrogen atmosphere, the initial temperature was 30° C.; the temperature increased at a rate of 10° C./minute. The results were compared with those obtained under the same conditions for the rimonabant crystal form II.

For each compound, the melting peak and the enthalpy change of the substance (ΔH) were measured before and after melting, in joules per gram of material.

According to FIG. 2, the crystal form II has a melting peak at 157±2° C. with ΔH=66±2 J/g.

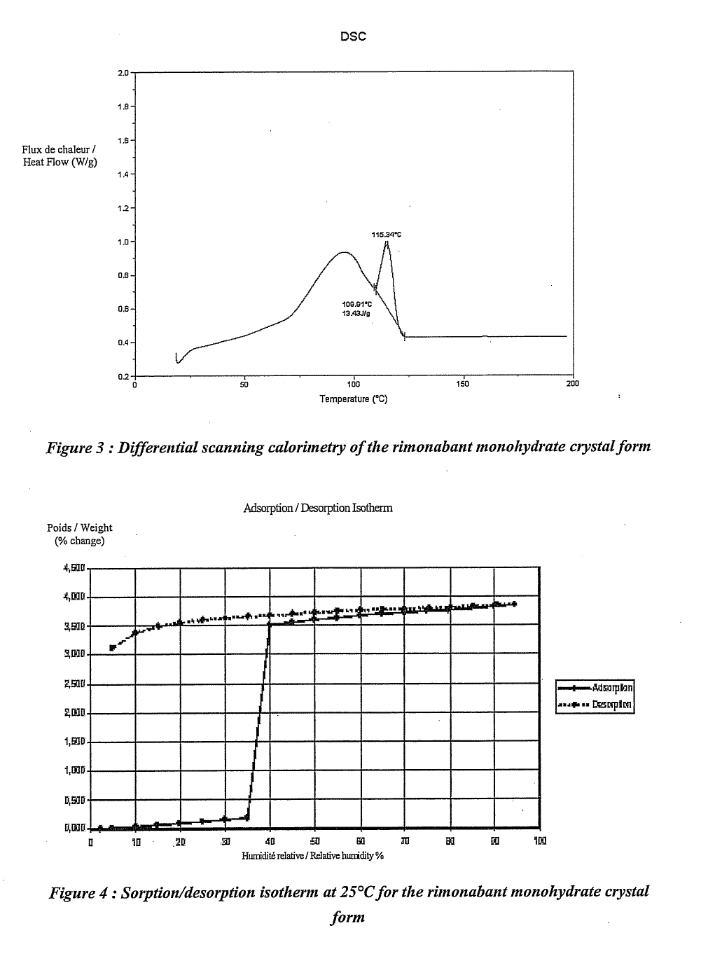

According to FIG. 3, the rimonabant monohydrate crystal form loses its water of crystallization molecule between 40° C. and 100° C. It has simultaneously a melting peak situated between 95° C.±5° C. and 115° C.±5° C.

Analysis of water vapor sorption/desorption measurements was carried out on the rimonabant monohydrate crystal form on a SGA100 analyzer sold by VTI (USA). It was operated between 0% and 100% relative humidity at 25° C. after degassing the monohydrate form at 80° C. for 3 hours. The rimonabant monohydrate loses its water of hydration molecule during drying at 80° C. During the water vapor sorption cycle, the conversion of the rimonabant into rimonabant monohydrate occurs starting from 40% relative humidity. The sorption/desorption isotherm is represented in FIG. 4.

According to the present invention, the rimonabant monohydrate crystal form was also characterized by its infrared (IR) spectrum. This was compared with that of the rimonabant crystal form II described previously.

The infrared (IR) spectra of these 2 rimonabant crystal forms were recorded on Perkin Elmer System 2000 FT-IR, spectrophotometers, between 400 cm⁻¹ and 4000 cm⁻¹, with a resolution of 4 cm⁻¹, in a potassium bromide disc, the compound tested being at a concentration of 0.5% by weight.

These spectra are characterized by the absorption bands given in Tables 1 and 2 below.

TABLE 1
IR spectrum, rimonabant monohydrate crystal form
λ (cm⁻¹)

3637
3385
1658
1554
1496
1264
990
918
780

TABLE 2
IR spectrum, rimonabant crystal form II
λ (cm⁻¹)

3311
2787
1683
1526
1484
986
922
781

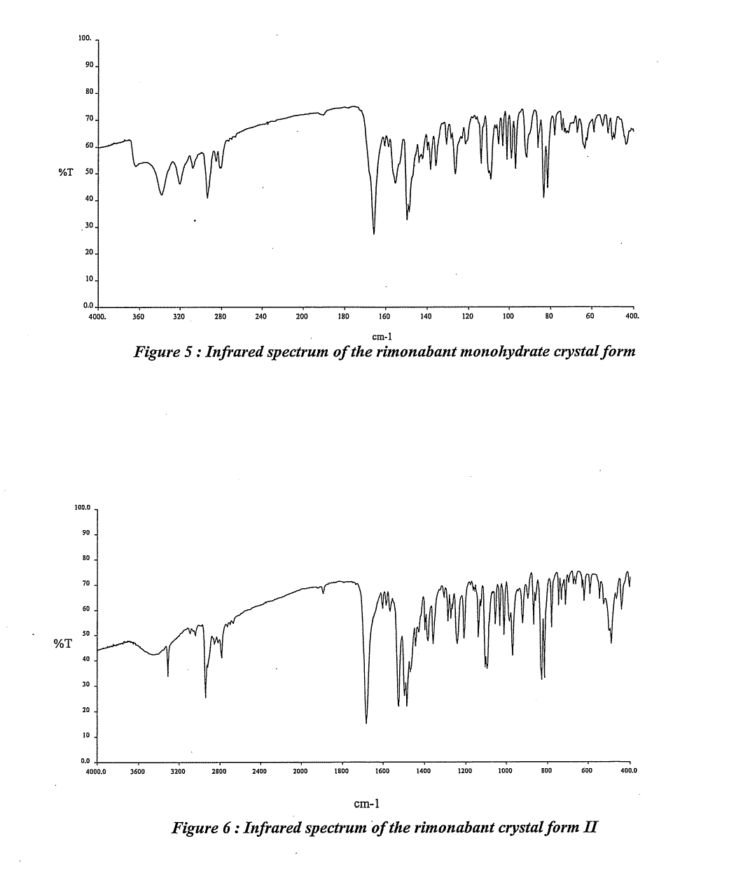

The wide band observed from 3637 to 3208 cm⁻¹ in the IR spectrum of the rimonabant monohydrate crystal form (FIG. 5) corresponds to the vibration of the H—O—H bonds of the hydrate and makes up one of the features of said IR spectrum.

For the remainder of the IR spectra, by comparing FIGS. 5 and 6 as they are shown, slight differences are observed in the positions and/or intensities of the lines, but the 2 spectra have the same general appearance.

Thus the IR spectrum of the rimonabant monohydrate crystal form is characterized by the following absorption bands: λ (cm¹)=3637; 3385; 1658; 1554; 1496; 990; 780 and more particularly by the bands λ=3637 cm⁻¹; 3385 cm⁻¹; 1658 cm⁻¹; 1554 cm⁻¹ and 1496 cm⁻¹.

The rimonabant monohydrate crystal form is also characterized by the characteristic lines of the powder X-ray diffractogram.

The powder X-ray diffraction profile (diffraction angle) was determined with a Bragg-Brentano Siemens D500TT (theta/theta) diffractometer; CuKα₁ source, λ=1.5406 Å; scanning range 2° to 40° at 1° per minute in 2 theta Bragg angle.

The characteristic lines of the diffractogram are given in Table 3 below:

TABLE 3
Powder X-ray, rimonabant monohydrate crystal form

| Peak | Angle |
|---|---|
| Ångströms | 2-Theta° |
| d = 9.049 | 9.311 |
| d = 8.35 | 10.586 |
| d = 6.501 | 13.610 |
| d = 4.964 | 17.854 |
| d = 4.167 | 21.307 |

Under the same conditions, the characteristic lines of the powder X-ray diffractogram of the rimonabant crystal form II was recorded, the characteristic lines are given in Table 4 below:

TABLE 4
Powder X-ray, rimonabant crystal form II

| Peak | Angle |
|---|---|
| Ångströms | 2-Theta° |
| d = 17.41664 | 5.070 |
| d = 8.70963 | 10.148 |
| d = 8.19062 | 10.793 |
| d = 5.82785 | 15.191 |
| d = 4.63425 | 19.136 |
| d = 3.49212 | 25.486 |

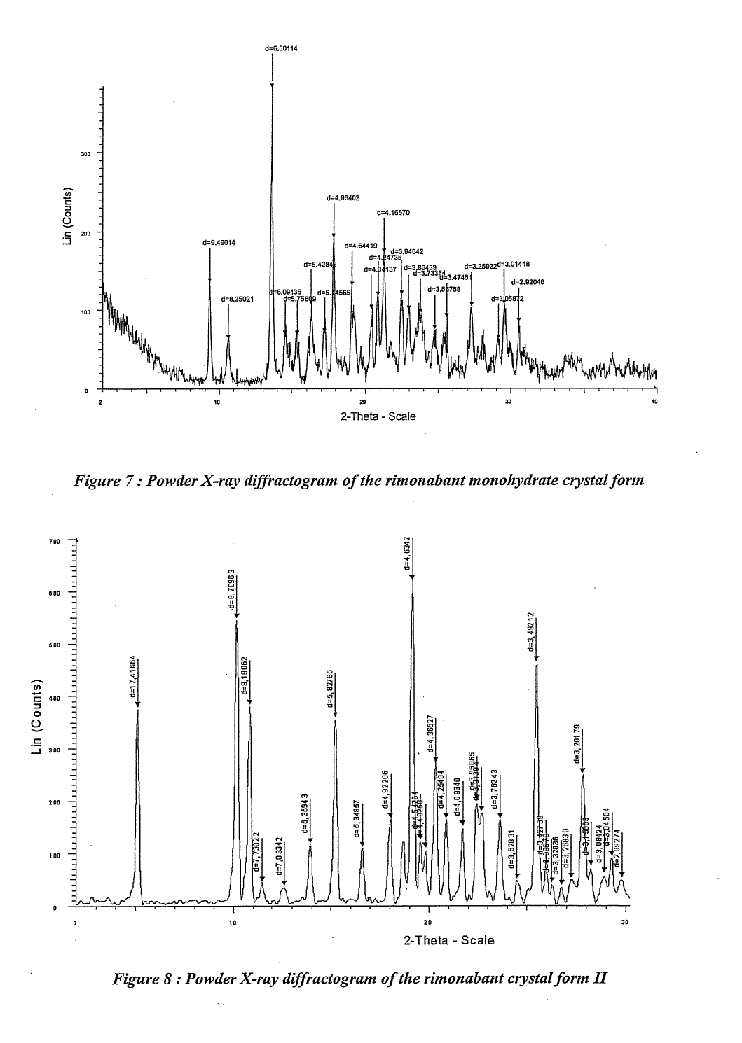

The corresponding diffractograms are reproduced in FIGS. 7 and 8. The rimonabant monohydrate crystal form was also characterized by its crystal structure, for which the lattice parameters were determined by single-crystal X-ray diffraction.

TABLE 5
Lattice parameter, rimonabant monohydrate crystal form

| Molecular formula | C₁₃N₄O₂C₂₂H₂₃ | |
|---|---|---|
| Molecular weight | 481.79 | |
| Lattice structure | triclinic | |
| Space group | P-1 | |
| Lattice parameter a | 7.424 | (2) Å |
| Lattice parameter b | 13.223 | (3) Å |
| Lattice parameter c | 24.718 | (6) Å |
| Lattice parameter α | 96.89 | (1)° |
| Lattice parameter β | 96.17 | (1)° |
| Lattice parameter γ | 90.66 | (1)° |
| Cell volume | 2394 | (1) Å³ |
| Number of molecules per cell: Z | 4 | |
| Calculated density | 1.336 | g/cm³ |

The values in brackets in the right-hand column correspond to the standard deviations observed for these measurements.

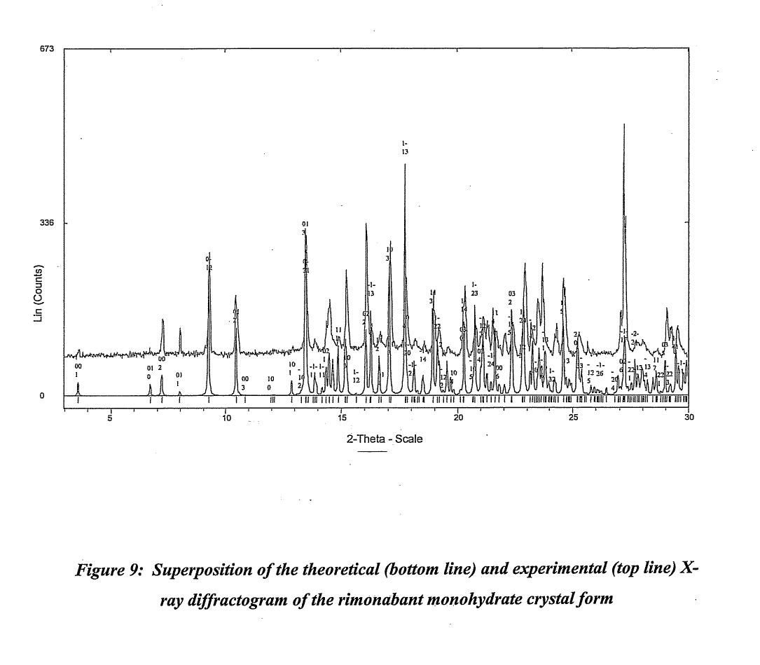

In FIG. 9, the theoretical and experimental diffractograms of rimonabant monohydrate are compared by superposition.

From the lattice parameters and the x, y, z atomic coordinates of the atoms of the molecule, computer software was used to plot projections of the crystal unit cell for the molecule concerned.

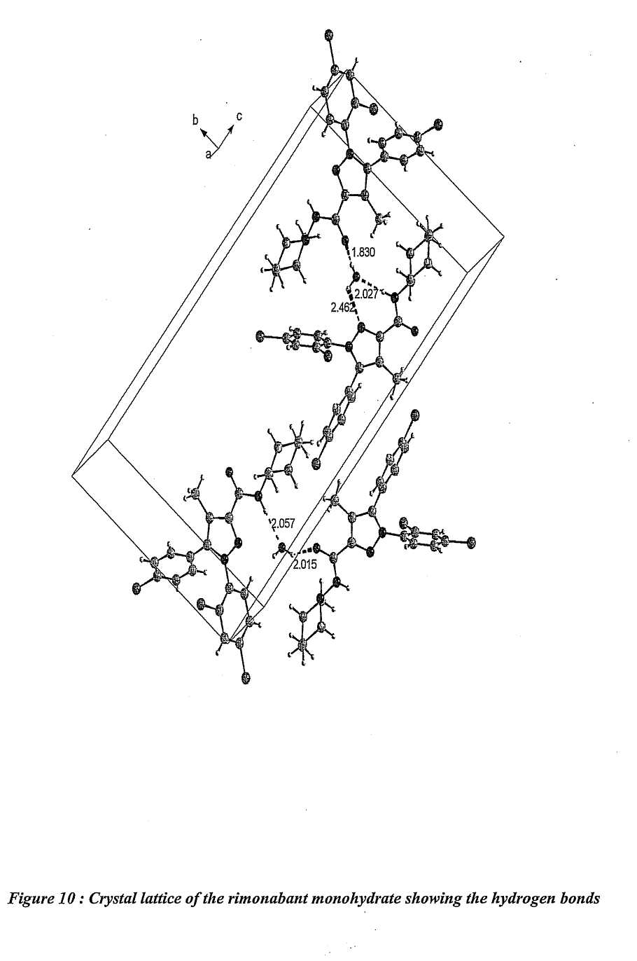

As can be seen in FIG. 10, this representation of the molecule in the crystal unit cell demonstrates the presence of the water molecule (water of crystallization) that indeed participates in the crystal structure.

### EXAMPLE

### Preparation of the Rimonabant Monohydrate Crystal Form

80 g of rimonabant form II were suspended in 400 ml of acetone at room temperature, with stirring, overnight. The suspension was filtered so as to obtain a saturated clear solution of rimonabant in acetone. 100 ml of water were introduced into this solution, which caused progressive insolubilization of the rimonabant monohydrate in crystal form. The suspension obtained was cooled to 5° C., then filtered. The product was dried at room temperature for 48 hours.

65 g of the expected compound were obtained, the water content of which was 3.4%, which was consistent with the theoretical water content (3.7%).

The rimonabant content of the compound obtained was 96.6%. Thus, it appears that there are no quantifiable impurities in the compound obtained.

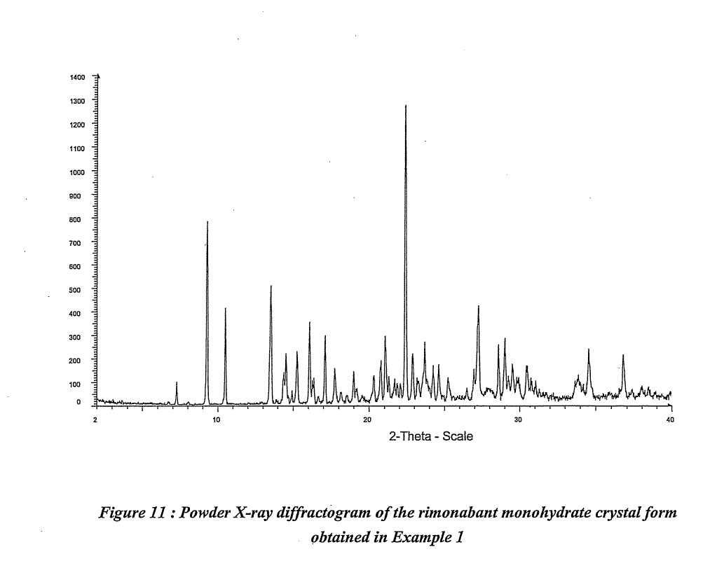

The powder X-ray diagram is shown in FIG. 11.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A substantially pure rimonabant monohydrate.

2. A crystal form of the rimonabant monohydrate as claimed in claim 1, which exhibits melting peak between 95° C.±5° C. and 115° C.±5° C.

3. A crystal form of the rimonabant monohydrate as claimed in claim 1, which exhibits infrared spectrum absorption bands as listed below:
λ (cm⁻¹)
3637
3385
1658
1554
1496
1264
990
918
780

4. A crystal form of the rimonabant monohydrate as claimed in claim 1, which exhibits infrared spectrum absorption bands at λ (cm⁻¹)=3637; 3385; 1658; 1554 and 1496.

5. A crystal form of the rimonabant monohydrate as claimed in claim 1, which exhibits the powder X-ray diffractogram lines described below:

| Peak | Angle |
|---|---|
| Ångströms | 2-Theta° |
| d = 9.049 | 9.311 |
| d = 8.35 | 10.586 |
| d = 6.501 | 13.610 |
| d = 4.964 | 17.854 |
| d = 4.167 | 21.307 |

6. A crystal form of the rimonabant monohydrate as claimed in claim 1 having the following molecular formula and molecular weight and which exhibits the lattice parameters described below:

| Molecular formula | C₁₃N₄O₂C₂₂H₂₃ | |
|---|---|---|
| Molecular weight | 481.79 | |
| Lattice structure | triclinic | |
| Space group | P-1 | |
| Lattice parameter a | 7.424 | (2) Å |
| Lattice parameter b | 13.223 | (3) Å |
| Lattice parameter c | 24.718 | (6) Å |
| Lattice parameter α | 96.89 | (1)° |
| Lattice parameter β | 96.17 | (1)° |
| Lattice parameter γ | 90.66 | (1)° |
| Cell volume | 2394 | (1) Å³ |
| Number of molecules per cell: Z | 4 | |
| Calculated density | 1.336 | g/cm³ |

7. A method for preparing the rimonabant monohydrate as claimed in claim 1, which comprises:
(a) dissolving rimonabant in an organic solvent; and
(b) adding water to the resulting solution.

8. The method as claimed in claim 7, wherein:
(a) the organic solvent in Step (a) is selected from the group consisting of:
methylcyclohexane;
acetonitrile;
4-methyl-2-pentanone;
acetone;
toluene;
dimethylsulfoxide; and
a mixture thereof; and
(b) water is added drop by drop in Step (b).

9. The method as claimed in claim 7, wherein:
(a) a saturated solution of rimonabant is prepared at room temperature in Step (a) using the solvent selected from the group consisting of:
methylcyclohexane;
acetonitrile;
4-methyl-2-pentanone;
acetone;
toluene;
dimethylsulfoxide; and
a mixture thereof;
(b) water is added drop by drop in Step (b); and
(c) separating the rimonabant monohydrate formed in Step (b).

10. The method as claimed in claim 9, wherein:
in Step (a), a solution of rimonabant in acetone is prepared; and
in Step (b), water is added drop by drop so as to obtain an acetone/water mixture containing between 10 and 30% of water by volume.

11. The method as claimed in claim 9, wherein:
in Step (a), a solution containing between 150 and 200 g/l of rimonabant in acetone is prepared.

12. The method as claimed in claim 7, wherein:
(a) the organic solvent in Step (a) is selected from the group consisting of:
acetonitrile;
4-methyl-2-pentanone;
acetone;
dimethylsulfoxide; and
a mixture thereof;
(b) water is added drop by drop in Step (b); and
(c) cooling the resulting mixture from Step (b) to a temperature between 0° and 15° C.

13. The method as claimed in claim 12, wherein:
(a) a saturated solution of rimonabant is prepared at room temperature in Step (a) using the solvent selected from the group consisting of:
acetonitrile;
4-methyl-2-pentanone;
acetone;
dimethylsulfoxide; and
a mixture thereof;
(b) water is added drop by drop in Step (b);
(c) cooling the resulting mixture from Step (b) to a temperature between 0° C. and 15° C.; and
(d) filtering the crystals formed therefrom.

14. The method as claimed in claim 12, wherein in Step (a) a saturated solution of rimonabant is prepared at room temperature in acetone.

15. The method as claimed in claim 12, wherein in Step (a), a mixture containing between 150 and 200 g/l of rimonabant in acetone is prepared at room temperature; and
in Step (b), between 10% and 30% of water by volume is added drop by drop.
